(19) United States
(12) Patent Application Publication
(10) Pub. No.: US 2017/0246233 A1
(43) Pub. Date: Aug. 31, 2017

(54) METHOD OF DECREASING BLOOD SUGAR

(71) Applicant: Chung Shan Medical University, Taichung City (TW)

(72) Inventors: Shoei-Yn Lin-Shiau, Taichung City (TW); Jen-Kun Lin, Taichung City (TW)

(73) Assignee: Chung Shan Medical University, Taichung City (TW)

(21) Appl. No.: 15/054,319

(22) Filed: Feb. 26, 2016

(51) Int. Cl. A61K 36/82; A61K 31/353; A61K 31/715; A61K 31/7012; A61K 9/00

(52) U.S. Cl. CPC A61K 36/82; A61K 31/7012; A61K 9/0053; A61K 31/715; A61K 31/353; A61K 2236/19

(57) ABSTRACT

The invention relates to a method of decreasing blood sugar by inhibition of α-glucosidase activity in a subject in need thereof, comprising administering pu-erh tea polysaccharides (PTPS) to the subject, wherein the active ingredients in PTPS are neutral sugar, protein, polyphenol, and uronic acids.

### FIELD OF THE INVENTION

The present invention relates to a method of decreasing blood sugar by inhibition of α-glucosidase activity in a subject in need thereof, comprising administering pu-erh tea polysaccharides (PTPS) to the subject, wherein the active ingredients in PTPS are neutral sugar, protein, polyphenol, and uronic acids.

### BACKGROUND OF THE INVENTION

Diabetes mellitus is a metabolic syndrome characterized by deleterious glucose uptake and hyperglycemia. Type 2 diabetes is mainly induced by environmental factors such as being overweight and decreased physical activity. Imbalanced energy metabolism such as β-cell dysfunction and peripheral insulin resistance results in type 2 diabetes. Type 2 diabetes is a major metabolic disorder worldwide. Therefore, investigation of the underlying molecular mechanism of type 2 diabetes and finding molecules to cure type 2 diabetes are very important issues in molecular medicine.

Starch is the most important source of carbohydrates for human beings and several animals. Meanwhile, α-amylase and α-glucosidase are digestive enzymes for carbohydrates. Salivary and pancreatic α-amylase cleave the α-(1→4)-_{D}-glycosidic bonds of starch at random sites, forming smaller oligosaccharides or disaccharides. α-Glucosidase is located in the brush border of the small intestine. α-Glucosidase hydrolyzes the terminal nonreducing α-1→4 linkage of oligosaccharides or disaccharides to release glucose molecules. One method of decreasing postprandial hyperglycemia is to suppress the release of glucose after consumption of a carbohydrate meal by inhibiting α-glucosidase. Acarbose is an α-glucosidase inhibitor that attenuates type 2 diabetes, but it is usually accompanied by many side effects such as abdominal distention, flatulence, diarrhea and meteorism. These serious side effects may be caused by significant inhibition of α-amylase. The bacteria residing in the colon can ferment undigested starch to release gas and low molecular weight substances. This abnormal fermentation results in side effects as mentioned above. Thus, α-glucosidase inhibitors that have fewer inhibitory effects on α-amylase may be promising agents to prevent postprandial hyperglycemia resulting from type 2 diabetes.

Tea is the most common beverage worldwide because of its flavor, taste and biological activity. Teas are classified into three categories according to the degree of fermentation: non-fermented teas, such as green tea; partially fermented teas, such as oolong tea; and fully fermented teas, such as black and pu-erh tea. Tea contains polyphenols including (−)-epigallocatechin 3-gallate (EGCG), (−)-epigallocatechin (EGC), (−)-epicatechin 3-gallate (ECG), (−)-epicatechin (EC), (+)-gallocatechin (GC) and (+)-catechin (C). During the fermentation process, tea polyphenols are oxidized to quinone and then condensed to form bisflavanol, theaflavin, thearubigen and other high molecular weight compounds.

Tea polysaccharide (TPS) is one of main components in tea extract. TPS is extracted from tea leaves. The components and activities of TPS have been studied in the past few years.

Oxidative stress is produced under diabetic conditions in various tissues and plays an important role in the development of complications, such as pancreatic β-cell dysfunction in diabetes. Glucose oxidation, the nonenzymatic glycation of proteins, may cause the formation of free radicals in diabetes. High levels of free radicals result in oxidative stress. Oxidative stress damages cellular organelles, increases lipid peroxidation and causes insulin resistance. Therefore, prevention of oxidative stress may be a potential method to avoid type 2 diabetes.

### BRIEF DESCRIPTION OF THE DRAWINGS

FIG. 1 shows the effects of Feng huang dancong (woolong tea, lightly fermented) and Dahongpao (woolong tea, heavily fermented) polysaccharides on α-glucosidase activity.

FIG. 2 shows the effects of different ages of pu-erh tea polysaccharides on α-glucosidase activity.

FIG. 3 shows the inhibitory effects of Acarbose on α-glucosidase and α-amylase; FIG. 3A shows the effect of acarbose on α-glucosidase activity; FIG. 3B shows the inhibition kinetics effects of different acarbose concentrations on α-glucosidase activity; FIG. 3C shows the effect of acarbose on α-amylase activity.

FIG. 4 shows the effects of different concentrations of Feng huang dancing, Dahongpao, and 1-, 3-5-year-old pu-erh tea polysaccharides on α-amylase activity; FIG. 4A: 0-10 μg/ml of Feng huang dancing and Dahongpao; FIG. 4B: 0-10 μg/ml of 1-, 3-5-year-old pu-erh tea; FIG. 4C: 0-500 μg/ml of Feng huang dancing and Dahongpao; FIG. 4D: 0-500 μg/ml of 1-, 3-5-year-old pu-erh tea.

FIG. 5 shows effects of PTPSs on blood sugar in ICR mice; FIG. 5A mice were orally administered acarbose (5 mg/kg of body weight), PTPS-5 (5 mg/kg of body weight) or water (control) along with starch (2 g/kg of body weight); FIG. 5B mice were orally administered maltose (2 g/kg of body weight) along with acarbose (5 mg/kg of body weight), PTPS-5 (5 mg/kg of body weight) or water (control); FIG. 5C mice were orally administered maltose (2 g/kg of body weight) along with acarbose (10 mg/kg of body weight), PTPS-5 (10 mg/kg of body weight) or water (control).

FIG. 6 shows the inhibition kinetics of different tea polysaccharides at different concentrations on α-glucosidase activity; FIG. 6A: inhibition kinetics of Feng huang polysaccharides on α-glucosidase activity; FIG. 6B: inhibition kinetics of Dahongpao polysaccharides on α-glucosidase activity. FIG. 6C: inhibition kinetics of 1-year-old pu-erh tea polysaccharides on α-glucosidase activity; FIG. 6D: inhibition kinetics of 5-year-old pu-erh tea polysaccharides on α-glucosidase activity.

### SUMMARY OF THE INVENTION

The present invention relates to a method of decreasing blood sugar by inhibition of α-glucosidase activity in a subject in need thereof, comprising administering pu-erh tea polysaccharides (PTPS) to the subject, wherein the active ingredients in PTPS are neutral sugar, protein, polyphenol, and uronic acids.

### DETAILED DESCRIPTION OF THE INVENTION

The detailed description provided below in connection with the appended drawings is intended as a description of the present examples and is not intended to represent the only forms in which the present example is constructed or utilized. The description sets forth the functions of the examples and the sequence of steps for constructing and operating the examples. However, the same or equivalent functions and sequences may be accomplished by different examples.

The terms “a”, “an”, and “the” as used herein are defined to mean “one or more” and include plural referents unless the context clearly dictates otherwise.

The present invention relates to a method of decreasing blood sugar by inhibition of α-glucosidase activity in a subject in need thereof, comprising administering pu-erh tea polysaccharides (PTPS) to the subject, wherein the active ingredients in PTPS are neutral sugar, protein, polyphenol, and uronic acids.

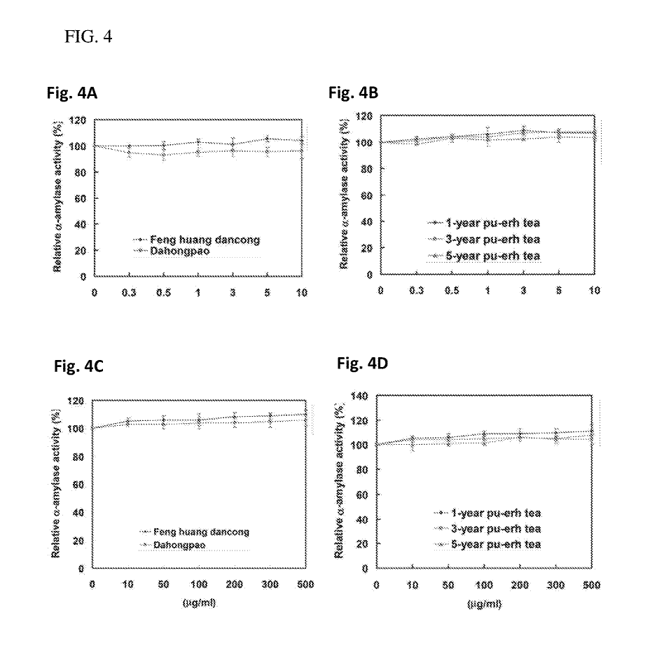

It is interesting to see that TPS from young pu-erh tea (1 and 3 years old) show nonsignificant inhibition of α-amylase, whereas TPS from 5 years old pu-erh tea show moderate inhibition of α-amylase (FIG. 4B and FIG. 4D). The higher content of polyphenol and uronic acids (23%, Table 1) may cause these different effects.

Furthermore, the IC₅₀ of polyphenol from 5-year-old pu-erh tea on α-glucosidase activity is 7.58 μg/ml. The effective dose for PTPS-5 on inhibition of α-glucosidase activity is much lower than acarbose; therefore pu-erh tea polysaccharides can act as an adjunctive or substitute treatment for type 2 diabetes.

Type 2 diabetes has been a common metabolic disorder in recent years. Patients of type 2 diabetes have little sensitivity to insulin and cannot attenuate blood sugar after a meal. Therefore, finding an effective way to prevent or cure type 2 diabetes is very important. Because that α-glucosidase hydrolyzes the terminal non-reducing α-1→4 linkage of oligosaccharides or disaccharides to release glucose molecules, decreasing the activity of α-glucosidase may attenuate glucose release after a meal.

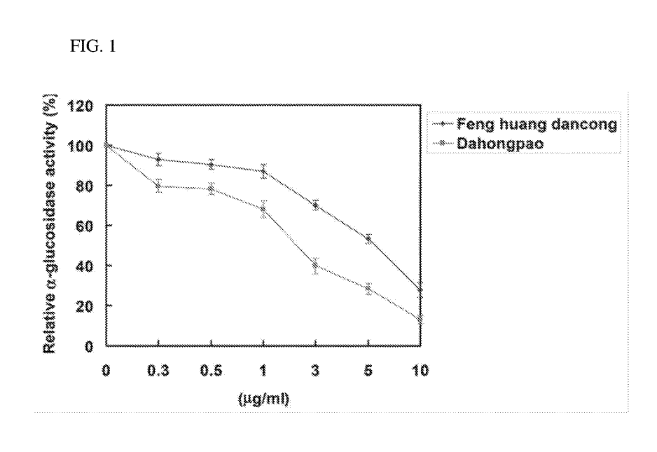

The present invention determines the inhibitory effects of Feng huang dancing (light in fermentation) and Dahongpao (semi-fermented tea) on α-glucosidase. As shown in FIG. 1, tea with a higher degree of fermentation has a higher inhibitory effect on α-glucosidase activity.

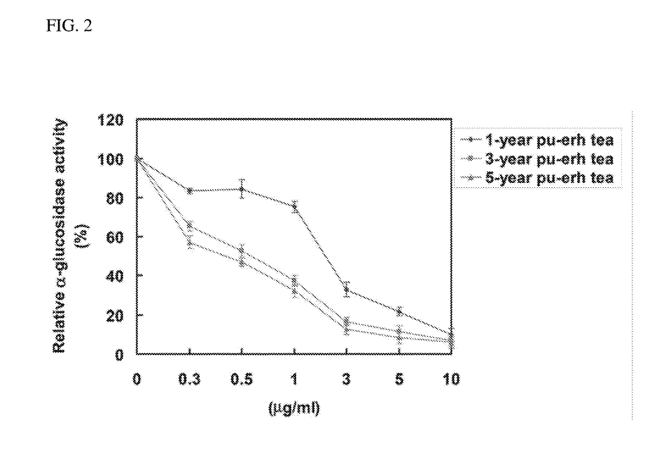

The present invention uses the TPSs extracted from pu-erh tea that had been aged for 1 year (PTPS-1), 3 years (PTPS-3) and 5 years (PTPS-5) to determine the effect on α-glucosidase activity. In a preferred embodiment, the PTPS is extracted from 5-year-aged Pu-erh tea. As shown in FIG. 2, PTPS-1, PTPS-3 and PTPS-5 attenuate α-glucosidase activity and the IC₅₀s are 2.192 μg/ml, 0.583 μg/ml and 0.438 μg/ml, respectively (Table 2).

TABLE 1
PTPSs content percentage

| Tea samples | Neutral sugar^{a} | Protein^{b} | Polyphenol^{c} | Uronic acids^{d} |
|---|---|---|---|---|
| Feng huang dancong | 14.839 ± 0.209 | 4.183 ± 0.397 | 5.835 ± 0.707 | 12.75 ± 0.034 |
| Dahongpao tea | 22.450 ± 4.838 | 4.729 ± 0.775 | 8.335 ± 0.400 | 13.19 ± 0.202 |
| 1-year old pu-erh tea | 20.381 ± 1.986 | 9.359 ± 1.937 | 8.335 ± 2.828 | 11.89 ± 0.317 |
| 3-year old pu-erh tea | 21.208 ± 2.404 | 3.907 ± 0.969 | 7.835 ± 2.828 | 21.95 ± 0.803 |
| 5-year old pu-erh tea | 24.519 ± 0.314 | 5.551 ± 1.356 | 14.335 ± 2.828 | 23.10 ± 1.060 |

Therefore, the present invention investigates whether older pu-erh tea has a higher inhibitory effect on α-glucosidase than younger pu-erh tea. PTPS-3 has a higher inhibitory effect on α-glucosidase than PTPS-1, and PTPS-5 has a higher inhibitory effect on α-glucosidase than PTPS-3 (FIG. 2).

In a preferred embodiment, the mass fraction (w/w) percentages of neutral sugar, protein, polyphenol, and uronic acids in tea are 12.3-36.8%, 2.8-8.3%, 7.2-21.5%, and 11.6-34.7%, respectively.

In another preferred embodiment, the mass fraction (w/w) percentages of neutral sugar, protein, polyphenol, and uronic acids in tea are 24.2-24.8%, 4.2-6.9%, 11.5-17.2%, and 22.0-24.2%, respectively.

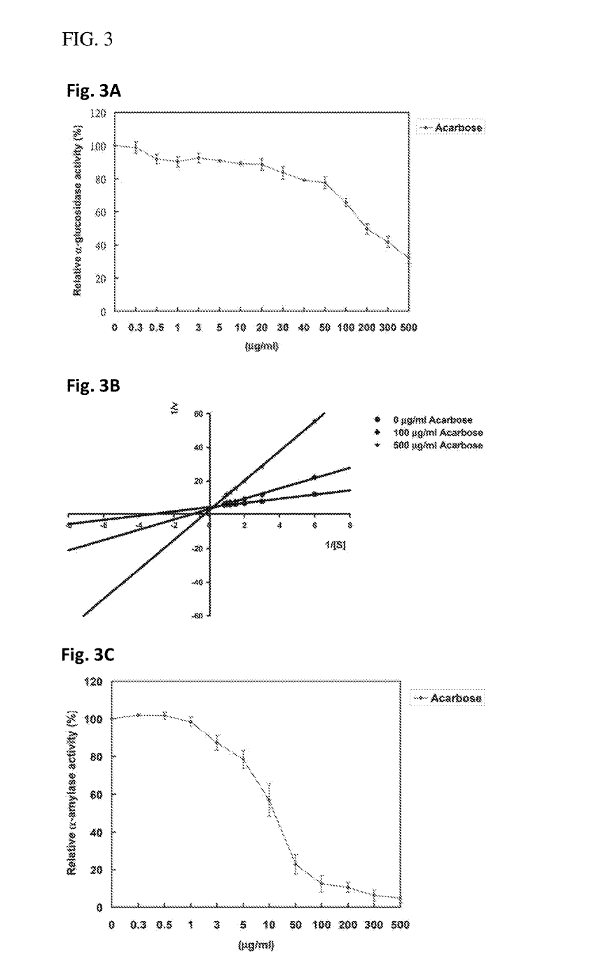

The clinical type 2 diabetes drug acarbose causes many side effects because it significantly inhibits α-amylase activity, resulting in abnormal fermentation by bacteria residing in the colon. In order to assess the remedial effects of PTPSs and acarbose on type 2 diabetes, the present invention compares their inhibitory effects on α-glucosidase and α-amylase activities. Acarbose indeed attenuates α-glucosidase activity (FIG. 3A) and the IC₅₀ is 207.195 μg/ml. The double reciprocal plot result indicates that acarbose has a competitive inhibitory effect on α-glucosidase (FIG. 3B). Acarbose can also reduce α-amylase activity, and the IC₅₀ is 17.825 μg/ml (FIG. 3C).

PTPSs do not inhibit α-amylase activity either with lower (FIG. 4A) or higher concentrations (FIG. 4B). The IC₅₀ of acarbose on α-glucosidase activity is 207.195 μg/ml, which is much higher than the IC₅₀ of TPS on α-glucosidase activity (Table 1). The results suggest that PTPSs may be more appropriate than acarbose to control type 2 diabetes.

TABLE 2
Inhibition effects of acarbose and TPS on α-glucosidase and α-amylase

IC₅₀^{a} (μg ml⁻¹)

| Compounds | α-Glucosidase | α-Amylase |
|---|---|---|
| Feng huang dancong | 5.653 ± 0.061 | >500 |
| Dahongpao | 2.286 ± 0.005 | >500 |
| 1-year old pu-erh tea | 2.192 ± 0.005 | >500 |
| 3-year old pu-erh tea | 0.583 ± 0.032 | >500 |
| 5-year old pu-erh tea | 0.438 ± 0.004 | >500 |
| Acarbose^{b} | 207.195 ± 41.288 | 17.825 |

According to International Diabetes Federation, the blood sugar level of a non-pregnant adult with diabetes should be under 180 mg/dL (10.0 mmol/L) in 1 hour after a meal to avoid chronic complications; and the blood sugar level of a healthy individual rises temporarily up to 140 mg/dL (7.8 mmol/L) shortly after a meal.

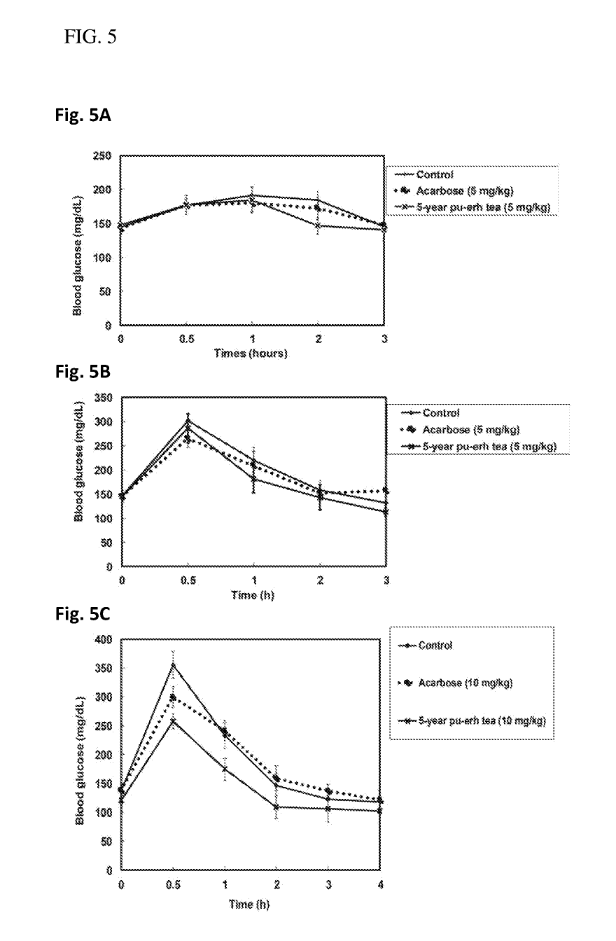

Oxidative stress is produced under diabetic conditions in various tissues. Preventing oxidative stress is important in antidiabetic treatments. PTPSs significantly revealed a scavenging effect on the superoxide radical. In one embodiment of animal model, the results indicate that PTPSs significantly slow down and attenuate blood sugar after feeding mice with starch, and PTPS-5 is more effective than acarbose at decreasing blood sugar (FIG. 5A). Feeding PTPS-5 along with maltose to mice gave a larger decrease in blood sugar after a meal than acarbose (FIG. 5B, FIG. 5C). In an embodiment, the dose of PTPS-5 is 5-10 mg/kg. In another embodiment, the dose of PTPS-5 is 1-20 mg/kg. The results suggest that the effect of decreasing blood sugar in a subject in need thereof is dose-dependent, which means higher PTPS dose is more effective on decreasing blood sugar.

According to the abovementioned results, PTPSs are better than acarbose at controlling diabetes by lowering down the blood sugar level to 180 mg/dL in one hour (FIG. 5).

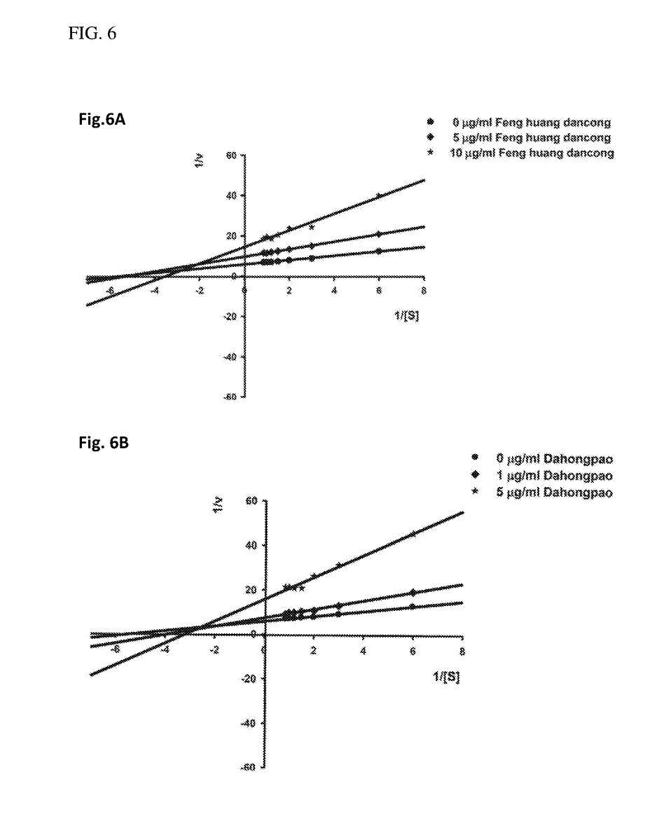

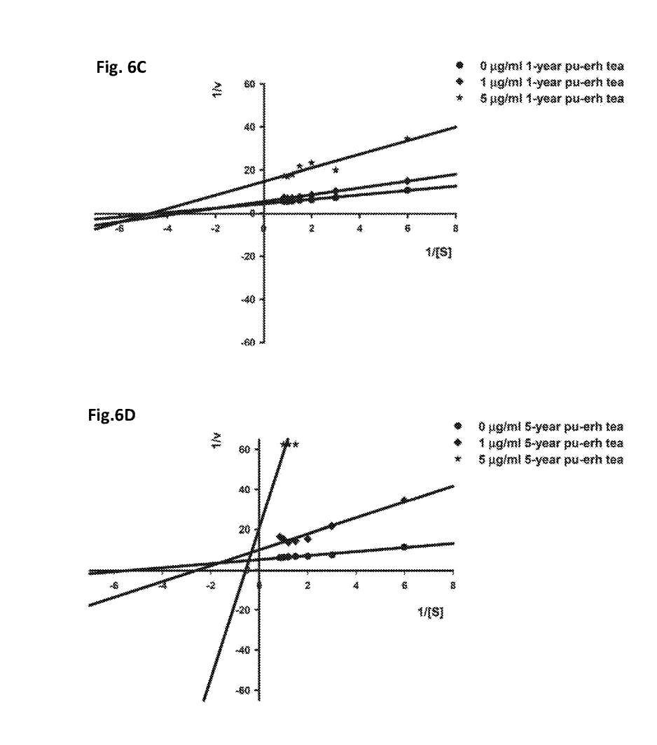

The present invention also uses various concentrations (0.167, 0.333, 0.5, 0.667, 0.833, 1 and 1.167 mM) of α-glucosidase substrate and different concentrations of TPSs to tests the inhibitory effects of TPSs on enzyme kinetics. As indicated from the Lineweaver-Burk double reciprocal plot (LBDRP) embodiment, Feng huang dancong, Dahongpao and pu-erh tea polysaccharides perform mixed-type inhibitory effects on α-glucosidase (FIG. 6A-FIG. 6D).

Comparing the effects of PTPSs and acarbose on α-glucosidase and α-amylase activities, PTPSs from Feng huang dancong, Dahongpao and pu-erh tea (1-year-old, 3-year-old and 5-year-old) significantly inhibit α-glucosidase activity and do not affect α-amylase activity. The IC₅₀ of acarbose and the IC₅₀s of TPSs from Feng huang dancong, Dahongpao and pu-erh tea (1-year-old, 3-year-old and 5-year-old) on α-glucosidase activity are 207.195 μg/ml, 5.653 μg/ml, 2.286 μg/ml, 2.192 μg/ml, 0.583 μg/ml and 0.438 μg/ml, respectively (Table 1). Clearly, the PTPSs have more significant effects on α-glucosidase than acarbose does (Table 2). In addition, the PTPSs do not diminish α-amylase activity but acarbose does (Table 2). The inhibition kinetics are illustrated in FIG. 6A, FIG. 6B, FIG. 6D and FIG. 3B, separately.

PTPS-5 (5 mg/kg of body weight) significantly helps the mice reduce their blood sugar after being fed (FIG. 5). Increasing the dosages of acarbose and PTPS-5, PTPS-5 (10 mg/kg of body weight) has a much higher effect on lowering blood sugar than acarbose (10 mg/kg of body weight) (FIG. 5C).

### Examples

The examples below are non-limiting and are merely representative of various aspects and features of the present invention.

Materials

Samples of Feng huang dancong, Dahongpao and different ages of pu-erh tea were kindly provided by Professor Y. Wang (Zhejiang University, Hangzhou, China). The α-glucosidase, isolated from Saccharomyces cerevisiae (Sigma Product No G 0660), is an exoglycosidase and hydrolyzes terminal, non-reducing α-1→4, α-1→3, and α-1→2 linked D-glucose residues from oligosaccharides, with a preference for the α-1→4 linkage. Relatively small substrates such as maltose and sucrose are cleaved more rapidly than larger substrates such as starch. α-Amylase, p-nitrophenyl alpha-_{D}-glucoside, methionine, riboflavin and EDTA were purchased from Sigma-Aldrich (St. Louis, Mo., USA).

Extraction and Isolation of Tea Polysaccharides (TPS)

Dried tea leaves (100 g) were mixed with 1 L of ethanol (80%, v/v) to remove most of the free form polyphenols and monosaccharides. After the supernatant was removed, the tea residues were dried in air and then extracted with hot water at 70° C. for 60 min (3 times). The aqueous extracts were concentrated and then precipitated with 4-volumes of 95% ethanol. The precipitate that formed was collected by centrifugation at 3000 g for 10 min and repeatedly washed sequentially with 50 mL of ethanol, acetone, and ether, 3 times. The precipitate was dissolved in hot water (70° C.) and dialyzed against distilled water for 48 h with dialysis tubing (molecular weight cutoff, 7000 Dalton) to remove low-molecular weight matter, and then concentrated and precipitated with 4 volumes of 95% ethanol. The isolated PTPSs were dissolved (water, 60° C.) and the remaining ethanol was removed on a rotary evaporator under reduced pressure. The product was then lyophilized to a powder.

Analysis of the Composition of PTPS

The neutral sugar content was measured by the anthrone-sulfuric acid method using glucose as a standard. The uronic acid content was determined by the m-hydroxydiphenyl method using galacturonic acid as a standard. Protein content was analyzed with BioRad solution using bovine serum album as the standard. Polyphenol content was determined by the ferrous tartrate method. The composition of PTPS is listed in Table 2. It is worth noting that rather high polyphenol from polysaccharide-polyphenol complex, and uronic acid contents were found in 5-year old pu-erh tea PTPS (Table 1).

Enzyme Assay of α-Glucosidase Activity

A reaction mixture containing 200 μl deionized water (only in the blank test), 5 ml of 67 mM potassium phosphate buffer (pH 6.8), 200 μl of 3 mM glutathione, and 200 μl of α-glucosidase enzyme solution (0.3 unit ml⁻¹, only in the sample tests) was equilibrated at 37° C. Then 500 μl of 10 mMp-nitrophenyl α-_{D}-glucoside solution was added and the mixture was incubated at 37° C. for 20 minutes. Two ml of test solution or blank solution were mixed with 8 ml of 100 mM sodium carbonate solution to stop the reaction. The test solution and blank solution are recorded at OD 400 nm.

The active portion of pu-erh tea polysaccharides is the polysaccharide, based on the fact that after treated with 0.1 N HCl (in a boiling water bath for 30 min) and α-amylase (1 unit ml⁻¹ for 6 h), the tea polysaccharide lost most (90%) of its inhibitory effect on α-glucosidase.

Enzyme Assay of α-Amylase Activity

Reaction mixtures containing 1 ml of 1% (w/v) soluble starch solution, 1 ml of α-amylase solution (1 unit ml¹) and 1 ml of sodium potassium tartrate solution were placed in a boiled water bath for 15 minutes, and then cooled to room temperature. Twenty mL of 1 M HCl was added to stop the enzyme reaction. Then the light blue color was developed by addition of iodine reagent (5 mM of iodine and 5 mM of KI), and finally 9 mL of water was added. The absorbances of the resulting solutions were read at 540 nm.

Animal Studies

The animal testing procedures and general handling complied with the ethical guidelines and standards established by the institutional animal care and use committee at National Taiwan University (most regulations followed the standard of the NIH (USA)).

Four-week-old male ICR mice weighing 18-20 g were purchased from the Laboratory Animal Center of National Taiwan University College of Medicine. Mice were randomly divided into groups (five mice per group), consisting of control, acarbose (5 mg/kg), TPS of 5-year-old pu-erh tea (5 mg/kg) and TPS of 5-year-old pu-erh tea (10 mg/kg) groups. After the mice were fasted overnight, starch (2 g/kg of body weight) or maltose (2 g/kg of body weight) was orally administered along with acarbose (5 mg/kg of body weight), PTPS-5 (5 or 10 mg/kg of body weight) or water (acted as a control). Blood glucose levels were measured at 0, 0.5, 1, 2 and 3 hours using a glucometer.

The above examples are merely illustrative to explain the principles and efficacy of the present invention, and are not intended to limit the present invention. Those skilled in the art will realize that changes and modifications may be made thereto without departing from the spirit of the invention, and it is intended to include all such modifications as fall within the true scope of the invention.

What is claimed is:

1. A method of decreasing blood sugar by inhibition of α-glucosidase activity in a subject in need thereof, comprising administering 5-year-aged pu-erh tea polysaccharides (PTPS-5) to the subject, wherein the active ingredients in PTPS-5 are neutral sugar, protein, polyphenol, and uronic acids.

2. The method of claim 1, wherein the active ingredient is polyphenol and uronic acid.

3. The method of claim 1, wherein the polyphenol is polysaccharide-polyphenol complex form.

4. The method of claim 1, wherein the dose of PTPS-5 is 1-20 mg/kg.

5. The method of claim 3, wherein the dose of PTPS-5 is 5-10 mg/kg

6. The method of claim 1, wherein the mass fraction (w/w) percentages of neutral sugar, protein, polyphenol, and uronic acids in tea are 12.3-36.8%, 2.8-8.3%, 7.2-21.5%, and 11.6-34.7%, respectively.

7. The method of claim 1, wherein the mass fraction (w/w) percentages of neutral sugar, protein, polyphenol, and uronic acids in tea are 24.2-24.8%, 4.2-6.9%, 11.5-17.2%, and 22.0-24.2%, respectively.

8. The method of claim 1, wherein the PTPS-5 is administered to the subject along with a meal.

9. The method of claim 1, further preventing prandial hyperglycemia in one hour.

10. The method of claim 7, wherein the PTPS-5 is administered to the subject orally.

11. A method of preventing prandial hyperglycemia in one hour after a meal by inhibition of α-glucosidase activity in a subject in need thereof, comprising administering 5-year-aged pu-erh tea polysaccharides (PTPS-5) to the subject along with the meal, wherein the active ingredients in PTPS-5 are neutral sugar, protein, polyphenol, and uronic acids.

12. The method of claim 11, wherein the active ingredient is polyphenol and uronic acid.

13. The method of claim 12, wherein the polyphenol is polysaccharide-polyphenol complex form.

14. The method of claim 11, wherein the dose of PTPS-5 is 1-20 mg/kg.

15. The method of claim 13 wherein the dose of PTPS-5 is 5-10 mg/kg.

16. The method of claim 11, wherein the mass fraction (w/w) percentages of neutral sugar, protein, polyphenol, and uronic acids in tea are 12.3-36.8%, 2.8-8.3%, 7.2-21.5%, and 11.6-34.7%, respectively.

17. The method of claim 11, wherein the mass fraction (w/w) percentages of neutral sugar, protein, polyphenol, and uronic acids in tea are 24.2-24.8%, 4.2-6.9%, 11.5-17.2%, and 22.0-24.2%, respectively.
